(19) United States
(12) Patent Application Publication
(10) Pub. No.: US 2010/0168412 A1
(43) Pub. Date: Jul. 1, 2010

(54) Continuous Neutralizer Mixer Reactor and a Continuous Process for Quenching Chlorination Reaction Mixture in Production of Chlorinated Sucrose

(75) Inventors: Rakesh Ratnam, Karnataka (IN); Sundeep Aurora, Karnataka (IN); Sanjiv Shankar Anand, Karnataka (IN); Subramaniyam, Karnataka (IN)

Correspondence Address: THE NATH LAW GROUP, 112 South West Street, Alexandria, VA, 22314, US

(73) Assignee: V.B. MEDICARE PVT. LTD, Karnataka (IN)

(21) Appl. No.: 12/226,566

(22) PCT Filed: Apr. 19, 2007
(86) PCT No.: PCT/IN2007/000150

§ 371 Date: Dec. 3, 2008

(30) Foreign Application Priority Data
Apr. 27, 2006 (IN) 662/MUM/2006

(51) Int. Cl. C07H 1/06; C07H 1/00; B01J 19/18

(52) U.S. Cl. 536/127; 536/124; 422/108

(57) ABSTRACT

An improved process of production of a chlorinated sugar is described comprising chlorination of a partially protected sugar, wherein quenching as well as neutralization of chlorinated reaction mass is carried out concurrently and continuously in a reactor which is a continuous mixer as well as quencher providing continuous mixing of chlorination reaction mixture and pH adjusting solution and also provides for continuous quenching and continuous removal of quenched chlorinated reaction mixture.

### TECHNICAL FIELD

The present invention relates to method of neutralization of the chlorinated reaction mixture as a continuous process in a novel neutralizer mixer reactor used in the production of halo (chlorinated) sugars including 1′-6′-Dichloro-1′-6′-DIDEOXY-β-Fructofuranasyl-4-chloro-4-deoxy-galactopyranoside (TGS).

### BACKGROUND OF THE INVENTION

Strategies of prior art methods of production of 4,1′,6′ trichlorogalactosucrose (TGS) predominantly involve chlorination of sucrose-6-ester by use of Vilsmeier-Haack reagent derived from various chlorinating agents such as phosphorus oxychloride, oxalyl chloride, phosphorus pentachloride etc, and a tertiary amide such as dimethyl formamide (DMF) or dimethyl acetamide to chlorinate Sucrose-6-ester, to form 6 acetyl 4,1′,6′trichlorogalactosucrose. After the said chlorination reaction, the reaction mass is neutralized to pH 7.0-7.5 using appropriate alkali hydroxides of calcium, sodium, etc. to deesterify/deacetylate the 6 acetyl 4,1′,6′trichlorogalactosucrose to form 4,1′,6′ trichlorogalactosucrose (TGS).

After the said chlorination reaction, which takes place at elevated temperature, the reaction mass is highly acidic and has to be neutralized prior to purification and isolation of TGS. The neutralization of the chlorinated mass is carried out by addition of solution of hydroxides, carbonates and bicarbonates of alkali or alkaline earth metals. Also ammonia gas or various strength of ammonia solution can be used for neutralization.

This neutralization when carried out in the conventional reactor, several problems arise due to foaming, improper mixing of solution, improper temperature control and the like. These problems adversely affect the TGS content in the chlorinated mass during the neutralization stage. Hence the neutralization stage is a very crucial stage which needed to be controlled properly to ensure complete recovery of TGS from the chlorinated mass.

Further, during neutralization stage, in conventional methods, the volume of chlorinated mass increases to about 3-4 times of the original mass. Thus it becomes necessary that in any scale up of the reaction, the reactor of neutralization/quenching reactor should at least be three times that of the chlorination reactor. As the size of the reactor increases, the efficiency of temperature control, pH control and agitation comes down. It was found desirable to keep the increase in volume of the chlorinated reaction mass to minimum possible.

### SUMMARY OF THE INVENTION

In one embodiment of this invention, a continuous process is disclosed for neutralization of a chlorinated reaction mixture of partially protected chlorinated sugars carried out in a neutralizer mixer, the said neutralizer mixer provided with heat exchange mechanism for the purpose of concurrent quenching and continuous removal of the neutralized quenched mass.

In another embodiment of this invention, a neutralizer mixer is disclosed which provides a mixing arrangement for its liquid contents concurrently with quenching arrangement though a heat exchange mechanism simultaneously. In a further embodiment of this aspect of invention, an arrangement is provided to take out the quenched neutralized reaction mixture/reaction mass continuously from the mixer as fresh input of chlorinated reaction mixture and pH adjusting liquid is in progress. In a preferred embodiment of this aspect, an overflow is provided to the vessel holding the reaction mixture.

### BRIEF DESCRIPTION OF FIGURES

FIG. 1 shows schematic representation of an illustrative neutralizer mixer and accessories used for practicing the process of this invention, where (m) denotes a reactor vessel, (n) denotes the jacket of the reactor vessel, (o) denotes inlet for chlorinated sucrose, (p) denotes inlet for alkali, (q) denotes a temperature indicator, (r) denotes a pH sensor/controller, (s) denotes an overflow for neutralized and quenched reaction mass and (t) denotes a stirrer/agitator, (u) denotes a circulation pump, (v) denotes a heat exchanger, (w) denotes the brine inlet stream, (w′) denotes brine outlet stream, (x) denotes a collection vessel, (y) denotes the vessel containing alkali for neutralization, (z) denotes the vessel containing the chlorinated mass which has to be neutralized. It shall be clear to an ordinary person skilled in the art that the positions of various parts can be different than shown, and such of one or more of a variation is also included in this schematic diagram if the functions done and effects achieved by such variations are same as the one shown in this figure.

### DETAILED DESCRIPTION OF THE INVENTION

It shall be clear to a person skilled in the art that many variations of the description given in the following are possible intended to give the same result within the scope of the claims of this specification. Hence, following details are only illustrative of one or more ways of performing the invention and the description does not limit the scope of the claims. All obvious variations and adaptations falling within the scope of the claims with respect to reaction conditions, process conditions, specifications of equipment, dimensions of the equipment, design of the equipment, layout and capacities of the equipment claimed and used, layout of the equipment with respect to the disclosed details of the invention that are obvious to an ordinary skilled person are also included within the scope of this disclosure and claims.

A chlorinated reaction mixture as a process flow to which this invention is applicable may be a result of a part of one or more of a process of production of chlorinated sugar, disclosed in one or more of following citations: Fairclough, Hough and Richardson, Carbohydrate Research 40(1975) 285-298, Mufti et al (1983) U.S. Pat. No. 4,380,476, Rathbone et al (1986) U.S. Pat. No. 4,380,476, O'Brien et al (1988) U.S. Pat. No. 4,783,526, Tully et al (1989) U.S. Pat. No. 4,801,700, Rathbone et al (1989) U.S. Pat. No. 4,826,962, Simpson (1989) U.S. Pat. No. 4,889,928, Navia (1990) U.S. Pat. No. 4,950,746, Homer et al (1990) U.S. Pat. No. 4,977,254, Walkup et al (1990) U.S. Pat. No. 4,980,463, Neiditch et al (1991) U.S. Pat. No. 5,023,329, Vernon et al (1991) U.S. Pat. No. 5,034,551, Walkup et al (1992) U.S. Pat. No. 5,089,608, Dordick et al (1992) U.S. Pat. No. 5,128,248, Khan et al (1992) U.S. Pat. No. 5,136,031, Bornemann et al (1992) U.S. Pat. No. 5,141,860, Dordick et al (1993) U.S. Pat. No. 5,270,460, Navia et at (1994) U.S. Pat. No. 5,298,611, Khan et at (1995) U.S. Pat. No. 5,440,026, Palmer et at (1995) U.S. Pat. No. 5,445,951, Sankey (1995) U.S. Pat. No. 5,449,772, Sankey et al (1995) U.S. Pat. No. 5,470,969, Navia et al (1996) U.S. Pat. No. 5,498,709, Navia et al (1996) U.S. Pat. No. 5,530,106, Catani et al (2003) US patent application no. 20030171574, Ratnam et al (2005) WO/2005/090374, Ratnam et al (2005) WO/2005/090376 and the like. This is only an illustrative list, not claimed to be exhaustive and complete.

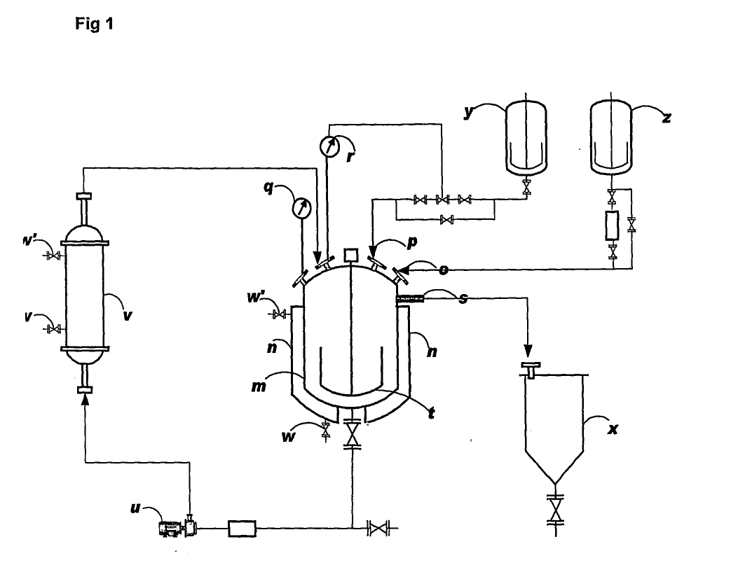

In a method of this invention, a novel neutralized mixer is provided that achieves neutralization as well as quenching simultaneously as a continuous process. The said neutralizer mixer and related equipment used for practicing this invention in a preferred embodiment are illustrated in FIG. 1. The said neutralizer mixer is provided with a vessel (m) to hold the fluid reaction mixture, an arrangement, preferably a stirrer (t), is provided to keep the contents of the vessel well mixed, one inlet (o) is provided for feeding chlorination reaction mixture and another (p) for fluid for pH adjustment simultaneously to the vessel. An online pH controller (r) is fitted to the vessel which monitors the actual pH of the fluid contents of the vessel and regulates a valve which controls addition of the pH adjusting liquid. The said pH adjusting liquid preferably used here is 7% ammonia. The vessel is provided with a jacket (n) through which a cooling liquid circulates, preferably brine. The vessel is provided with an outlet at bottom through which contents of the vessel are circulated through a heat exchanger (u) kept cool preferably at around 20° C. by circulating brine and the outlet of the heat exchanger returns the cooled reaction mixture to the vessel (m). Preferred method of removing neutralized and quenched mass continuously from the vessel is to provide an outlet (s). All the methods to achieve above functions and results covered by this invention may be achieved by alternative design too and all such alternative designs within the scope of the claims are covered by this invention.

Thus, in this invention, the chlorinated reaction mixture and 7% ammonia solution are added simultaneously and continuously in a reactor vessel provided with stirrer/agitator to achieve neutralization, the neutralized mass is simultaneously cooled in the vessel by brine maintained at 20° C. which circulates through a jacket surrounding the vessel, contents of the vessel are drawn from the bottom continuously by a pump which returns the contents to the vessel after passing through a heat exchanger which is kept cool to achieve further quenching of the reaction mixture by a brine maintained at 20° C. An overflow is provided through which excess of contents of neutralized and quenched reaction mixture passes out continuously for further processing for the purpose of recovery of TGS. This process achieves neutralization as well as quenching efficiently and the total volume of the neutralized and quenched mass is also substantially less than the total volume of neutralized and quenched reaction mass achieved in the conventional method.

The overflow of neutralized quenched reaction mass is further processed by one or more of purification and isolation for recovery of TGS. Purification may be done by one or more of a solvent extractive methods, or by chromatographic methods. Isolation of TGS is done by one or more of a methods of aqueous crystallization, solvent crystallization, spray drying, Agitated Thin Film drying and the like.

This system of a Continuous Neutralizer Mixer reactor reported here consists of a reactor where the neutralization takes place, a closed loop circulation of the neutralized mass from the reactor bottom through a heat exchanger for the purpose of controlling the temperature and the reactor also has an overflow from where continuously the neutralized solution flows out. The reactor is equipped with the pH sensor/controller which controls the inflow of the acid/alkali for pH adjustment in the reactor.

The chlorinated mass is fed into the reactor at a fixed flow rate and the solution for pH adjustment (hydroxides, carbonates and bicarbonates of alkali or alkaline earth metals, ammonia solution, etc) also flows simultaneously into the reactor and is mixed thoroughly. An online pH meter measures the pH as the solution gets mixed and a control system monitors the flow of the solution for pH adjustment. The neutralized mass continuously flows from the bottom of the reactor through a heat exchanger where the temperature is maintained and flows back to the reactor. This loop of passing through the heat exchanger is kept continuous and hence the temperature is well controlled in the reactor. The reactor is provided with an overflow at the top corner of the reactor through which the neutralized mass in the reactor continuously flows out and gets collected in a tank, which is taken for further purification.

Significant achievement of this inventive method of neutralization was that the typical size of a continuous neutralizer mixer required to handle the output from a chlorination reactor on a continuous basis was about half the size of the chlorination reactor. This resulted in a huge reduction in the size of the equipment meant for neutralization and also the utility requirement.

Throughout this specification, singular shall, unless context does not permit so, also include pleural of its own type and shall also include equivalents. Thus “A process of production of a chlorinated sugar” includes one or more of a process of production of chlorinated sugar; “a chlorinated sugar” includes one or more of a 4,1′,6′ trichlorogalactosucrose (TGS) 6,1′6′ trichlorogalactosucrose, 1′6′-dichloro, 1′4-dichloro, 4,6′-dichloro, 4,6,1′6′ tetrachloro derivatives and the like and so on.

### EXAMPLE 1

### Chlorination of Sucrose-6-Acetate Using Thionyl Chloride

400 L of DMF was charged into a Glass Lined reactor and 16 kg of carbon was added and mixed thoroughly. The nitrogen sparging was started and 344 L of thionyl chloride was added dropwise to the reactor. The temperature was maintained below 40° C. After the completion of addition of thionyl chloride, the mass was held at 35-40° C. for the reaction completion. Then the mass was cooled to 0-5° C. and 80 kg of 92% 6-acetyl sucrose in DMF (300 L) was added to it dropwise. The temperature was controlled below 5° C. and after the completion of addition of 6-acetyl sucrose, the mass was allowed to attain room temperature and was stirred at 30° C. for 3 hours. Then the mass was heated to 85° C. and maintained for 60 minutes and again heated to 100° C., maintained for 6 hours and further heated to 114° C. and maintained for 90 minutes.

The chlorinated mass was then cooled to 60° C. and was taken for neutralization.

### EXAMPLE 2

### Neutralization of Chlorinated Mass in Continuous Quenching System

150 L of DMF and 30 L of 25% ammonia solution in water was charged to the Continuous Neutralizer reactor. This solution was continuously circulated through the heat exchanger loop and was cooled to 10° C.

Addition of the chlorinated mass (˜950 L containing 28 kg 6-acetyl TGS) was started at a flow rate of 120 L/hr through a dip pipe arrangement. The reactor was also connected to a 7% ammonia solution tank through which the ammonia solution also was added simultaneously to the reactor. Temperature through the loop cycle was running continuously and was maintained at 20° C. The pH was monitored online and was controlled between 7.0-7.5 by the flow of the ammonia solution.

The quenched mass was collected through the overflow point provided in the reactor. The total quenched mass volume obtained was about 2500 L in 8 hrs. from a chlorinated reaction mass of 900 liters. The capacity of the neutralizer mixer reactor was 500 L. The quenched mass obtained by the method was analyzed for TGS content by HPLC. The overall efficiency of quenching was found to be 98% based on TGS content in the reaction mixture before and after quenching.

The quenched mass was then filtered to remove extraneous solids and then passed through affinity chromatography column containing Thermax ADS600 resin. The 6-acetyl TGS was adsorbed to the resin and the solution passed through the resin column as flow through. This flow through was taken up for DMF recovery. Then the resin was washed with water and then eluted out with 90% methanol and 10% ammonia solution.

The 6-acetyl sucrose was desorbed from the resin and as it passed out of the column, the deacylation also happened. The in-situ deacylation was completed during the product elution and was confirmed by TLC analysis.

The mass was then neutralized using dilute HCl and then taken for concentration. The concentrated mass was then again mixed with water up to a concentration of 3% TGS and was again passed through another column containing Thermax ADS 600 resin. The TGS was again adsorbed to the resin and the polar impurities were passed out in the flow through fractions. The adsorbed TGS was eluted out using 35% methanol in water and the non polar impurities remained bound to the column. The eluted fraction containing TGS was then concentrated. The concentrated mass was taken for water separation by azeotropic distillation using n-butanol and methanol.

After the water removal, the TGS was concentrated to 65% under vacuum and a temperature between 50-55° C. The solution was cooled from 55° C. to 30° C. in about 4-6 hours, then from 30° C. to 15° C. in about 2 hours and then further cooled to −5° C. in about 3.5 hours. The crystal slurry was then filtered and suck dried.

The wet solids obtained was then re-slurried in 5 L of ethyl acetate and stirred for 30 minutes at −5° C. Then the slurry was filtered and suck dried. Further the solids were dried in Vacuum Tray drier below 45° C.

The TGS crystals obtained were tested for purity and particle size. The purity was found to be 99.23% by HPLC and the overall yield obtained was 35% over sucrose input.

### EXAMPLE 3

### Comparison with Conventional Batch Quenching System

150 L of DMF and 500 L of 7% ammonia solution in water were charged to the Batch Neutralizer reactor. The temperature of the reactor was maintained at −5° C. The reactor was equipped with a pH sensor and the chlorinated mass (˜950 L containing 28 kg 6-acetyl TGS) was added. The reactor was kept under continuous stirring and the pH was maintained between 7.0 and 7.5. More 7% ammonia solution was added as and when the pH went acidic. The temperature in the reactor went up to 30° C. even with chilling up to −14° C. in the jacket. When the temperature was increased beyond 30° C., ice blocks were added inside the reactor to decrease the temperature.

The total quenched mass volume obtained was about 4500 L in 15 hrs. from a chlorinated reaction mass of 900 liters. The capacity of the neutralizer reactor was 5000 L. The quenched mass obtained by the method was analyzed for TGS content by HPLC. The overall efficiency of quenching was found to be 72%.

1. A process of production of a chlorinated sugar comprising chlorination of a partially protected sugar, wherein quenching as well as neutralization of chlorinated reaction mass is carried out concurrently and continuously in a reactor by addition of a pH adjusting liquid and the neutralized and quenched mass is also taken out of the reactor concurrently by one or more of a method.

2. A process of claim 1 wherein the said chlorinated reaction mass comprises mixture of one or more of a protected chlorinated sugar and one or more of a solvent comprising a tertiary amide, trichloro ethane, perchloroethylene, tolune, xylene and the like.

3. A process of claim 1 wherein the said chlorinated reaction mass and a pH adjusting liquid are added concurrently to a vessel of a reactor, preferably accompanied with stirring, and the resulting neutralized mixture is provided with one or more of a cooling arrangement.

4. A process of claim 3 wherein the said cooling arrangement comprises cooling by using circulation of brine maintained at a low temperature, preferably 20° C., the said circulation comprising circulating through a jacket (n) around the vessel and through a heat exchanger (v) where contents of the vessel circulate through the heat exchanger with help of a pump.

5. A process of claim 1, wherein:
a. the said chlorinated sugar comprises one or more of a 4,1′,6′ trichlorogalactosucrose (TGS) 6,1′6′ trichlorogalactosucrose, 1′6′-dichloro, 1′4-dichloro, 4,6′-dichloro, 4,6,1′6′ tetrachloro derivatives of sucrose and the like,
b. the said protected sugar comprising 6-acetyl-sucrose, 6-benzoyl sucrose, 6-lauryl sucrose, sucrose-6-propionate, sucrose-6-phthalate and the like,
c. the said tertiary amide comprising one or more of a dimethylformamide, dimethyl acetamide and the like,
d. the said pH neutralizing solution comprising an alkali, the said alkali further comprising one or more of ammonia, sodium hydroxide, potassium hydroxide, calcium hydroxide, barium hydroxide, sodium carbonate, sodium bicarbonate, carbonates, bicarbonates, sodium methoxide, calcium methoxide, potassium methoxide and the like,
e. the said method of removal outside the reactor comprising preferably by allowing an overflow,
f. the said reactor is preferably a continuous neutralizer/mixer comprising a reactor vessel (m),
i. a jacket (n) for flow of heat exchanging liquid,
ii. an inlet (o) for chlorinated reaction mass,
iii. an inlet (p) for alkali,
iv. a temperature indicator (q),
v. a pH sensor/controller (r),
vi. preferably having a stirrer/agitator (t). and
vii. an overflow (s) provided to take out neutralized and quenched reaction mass.

6. A process of claim 5 further comprising collecting the quenched and neutralized chlorinated reaction mass and subjecting it to a one or more of a method of purification and isolation of TGS.

7. A process of claim 5 wherein:
a. the chlorinated mass containing 6-acetyl TGS is added to a reactor vessel,
b. the said alkali is an ammonia solution in water, preferably around 7%, is added to the reactor concurrently,
c. temperature of the reaction mixture is maintained preferably at around 20° C. by controlling temperature of circulating brine, the circulation being through jacket of the reactor/vessel, through the jacket of heat exchanger (v) and the like,
d. the pH is controlled preferably between 7.0-7.5 by controlling flow of the said ammonia solution, and
e. the quenched mass is collected through the overflow point provided in the reactor.

8. A process of claim 6 wherein:
a. the quenched mass is filtered to remove extraneous solids,
b. the said filtered quenched mass is passed through affinity chromatography column containing preferably Thermax ADS600 resin to adsorb 6-acetyl TGS to the resin, rest of the solution is allowed to pass through the resin column as flow through,
c. the flow through, optionally, is taken up for DMF recovery,
d. the resin is washed with water, and then 6-acetyl TGS is eluted out preferably with 90% methanol and 10% ammonia solution accompanied by deacylation to TGS,
e. the mass is then neutralized using an acid preferably dilute HCl,
f. taken for concentration,
g. the concentrated mass is then taken for further purification and crystallization.

9. A neutralizer/mixer comprising a reactor vessel:
i. a jacket for flow of heat exchanging liquid,
ii. one or more of an inlet for adding reactants to the said vessel,
iii. a temperature indicator,
iv. a pH sensor/controller,
v. preferably having a stirrer/agitator,
vi. a circulation line from reactor bottom via pump through a heat exchanger and back to the reactor, and
vi. an overflow (s) provided to take out neutralized and quenched reaction mass.

10. A neutralizer/mixer comprising a reactor vessel (m),
i. a jacket (n) for flow of heat exchanging liquid,
ii. an inlet (o) for chlorinated reaction mass,
iii. an inlet (p) for alkali,
iv. a temperature indicator (q),
v. a pH sensor/controller (r),
vi. an overflow (s) for neutralized and quenched reaction mass, and
vii. preferably having a stirrer/agitator (t),
viii. a circulation line from reactor bottom of reactor via pump through a heat exchanger (v) and back to the reactor.
